(19) United States
(12) Patent Application Publication
(10) Pub. No.: US 2003/0219473 A1
(43) Pub. Date: Nov. 27, 2003

(54) Cochleates made with purified soy phosphatidylserine

(76) Inventors: Leila Zarif, Valbonne (FR); Feng Tan, Newark, NJ (US)

Correspondence Address: LAHIVE & COCKFIELD, 28 STATE STREET, BOSTON, MA, 02109, US

(21) Appl. No.: 10/304,567

(22) Filed: Nov. 26, 2002

Related U.S. Application Data
(63) Continuation of application No. 10/105,314, filed on Mar. 26, 2002.

(51) Int. Cl. A61K 9/127; A61K 31/7048; A61K 31/704; A61K 31/56; A61K 31/55; A61K 31/522

(52) U.S. Cl. 424/450; 514/27; 514/34; 514/169; 514/179; 514/182; 514/263.37; 514/217; 514/355; 514/288; 514/397; 514/420; 514/389; 514/449; 514/454; 514/627; 514/682; 514/291; 514/731

(57) ABSTRACT

Purified soy phosphatidylserine is used to make cochleates. The cochleates contain at least about 75% soy phosphatidylserine and optionally a bioactive load. A preferred cochleate contains the antifungal agent amphotericin B.

### FIELD OF THE INVENTION

The present invention relates to the ability of purified soy phosphatidylserine (PS) (PSPS) to make cochleates versus non-purified soy PS (NPSPS), to methods of preparing drug-cochleates from PSPS and to the use of this drug-loaded cochleate as a pharmaceutical treatment.

### BACKGROUND OF THE INVENTION

Cochleate delivery vehicles are a broad-based technology for the delivery of a wide range of bioactive therapeutic products. Cochleate delivery vehicles are stable phospholipid-cation precipitates composed of simple, naturally occurring materials, for example, phosphatidylserine and calcium.

The bilayer structure of cochleates provides protection from degradation for associated, or “encochleated,” molecules. Since the entire cochleate structure is a series of solid layers, components within the interior of the cochleate structure remain substantially intact, even though the outer layers of the cochleate may be exposed to harsh environmental conditions or enzymes. This includes protection from digestion in the stomach.

Taking advantage of these unique properties, cochleates have been used to mediate and enhance the oral bioavailability of a broad spectrum of important but difficult to formulate biopharmaceuticals, including compounds with poor water solubility, protein and peptide drugs, and large hydrophilic molecules. For example cochleate-mediated oral delivery of amphotericin B, large DNA constructs/plasmids for DNA vaccines and gene therapy, peptide formulations, and antibiotics such as clofazimine has been achieved.

Cochleates can be stored in cation-containing buffer, or lyophilized to a powder, stored at room temperature, and reconstituted with liquid prior to administration. Lyophilization has no adverse effects on cochleate morphology or functions. Cochleate preparations have been shown to be stable for more than two years at 4° C. in a cation-containing buffer, and at least one year as a lyophilized powder at room temperature.

Cochleates can be prepared by several methods, such as trapping or hydrogel methods. In the trapping method, the material to be formulated is added to a suspension of liposomes comprised mainly of negatively charged lipids. The addition of multivalent metal ions such as calcium (although other multivalent cations can be used), induces the collapse and fusion of the liposomes into large sheets composed of lipid bilayers which spontaneously roll up into cochleates. If desired, the cochleates can be purified to remove unencochleated material, then resuspended in a buffer containing multivalent metal ions.

The hydrogel method, U.S. Pat. No. 6,153,217, allows the preparation of nanocochleates having a particle size of less than one micron, which allows oral administration. The process disclosed in U.S. Pat. No. 6,153,217 involves an aqueous two-phase system of polymers where small unilamellar liposomes are added to a first polymer and then injected into a second polymer that is immiscible with the first polymer to create an aqueous two phase system of polymers. Nanocochleates are formed when a multivalent cation is added to the two phase system. The nanocochleates are useful for oral delivery of drugs. However, that patent did not disclose the use of purified soy PS in the preparation of cochleates.

Soy PS is sold in health food stores as a nutritional supplement. Non-purified (40%) PS has been used and studied as a nutritional supplement and as a component that has a beneficial effect on enhancing the brain functions in elderly people (Villardita C et al, Clin. Trials J. 24, 1987, 84-93).

Although non-purified soy PS (NSPS) has been sold and studied on patients, NSPS has never been studied and used to make cochleates and to deliver a drug using these cochleates.

It has been unexpectedly found that NPSP does not form cochleates and that a purification process is needed to enhance the NPSP in the content of PS, until at least about 75% by weight of PS is reached, such percentage allowing the formation of cochleates.

### SUMMARY OF THE INVENTION

Briefly, in accordance with the present invention, improved lipid based cochleates are made by using purified soy phosphatidylserine as the lipid source. The improved cochleates contain soy phosphatidylserine in an amount of at least about 75% by weight of the lipid. The improved cochleates can be empty or loaded cochleates. Loaded cochleates can contain any bioactive material or combination of bioactive materials such as, for example, proteins, small peptides, bioactive polynucleotides, an antiviral agent, an anesthetic, an antibiotic, an antifungal, an anticancer, an immunosuppressant, a steroidal anti-inflammatory, a non-steroidal anti-inflammatory, a tranquilizer, a nutritional supplement, an herbal product, a vitamin or a vasodilatory agent. Of particular interest in practicing the present invention, polyene antifungal agents are loaded into the present soy phosphatidylserine-based cochleates to provide a cost effective and improved antifungal drug with reduced toxicity. Preferred polyene antifungal agents include amphotericin-B and nystatin.

The improved lipid based cochleates of the present invention can be made by any means wherein soy phosphatidylserine is employed in an amount of at least about 75% by weight of the lipid component of the cochleate.

For example, soy phosphatidylserine/polyene cochleates are made by preparing small, unilamellar liposomes in an aqueous medium having a pH of between about 10 and about 12 wherein the liposomes have (i) a lipid bilayer comprising soy phosphatidylserine in an amount of at least about 75% by weight of the lipid bilayer and (ii) a load of polyene drug. A multivalent cation is added to the high pH liposomes to form the soy phosphatidylserine/polyene cochleates. The pH of the medium is then adjusted to about neutral and the soy phosphatidylserine/polyene cochleates are collected. The preferred polyene employed is amphotericin-B.

Another method of preparing the soy phosphatidylserine/polyene cochleates involves a two-phase aqueous polymer system where small, unilamellar liposomes are made in an aqueous medium having a pH of between about 10 and about 12 wherein the liposomes have (i) a lipid bilayer comprising soy phosphatidylserine in an amount of at least about 75% by weight of the lipid bilayer and (ii) a load of polyene drug. The liposomes are mixed with a first water soluble polymer to form a suspension. This suspension is then added to a suspension comprising a second water soluble polymer wherein the first and second polymers are immiscible thereby creating a two-phase polymer system. A multivalent cation is added to the two-phase polymer system to form the soy phosphatidylserine/polyene cochleates which are then collected.

The soy phosphatidylserine/polyene cochleates are then administered to patients with fungal infections. The present soy phosphatidylserine/polyene cochleates are conveniently administered orally even in the treatment of systemic fungal infections of immune compromised patients. The present phosphatidylserine/polyene cochleates are also administered parenterally, or by other means of administration. The preferred polyene is amphotericin-B.

### BRIEF DESCRIPTION OF THE FIGURES

FIG. 1A is an HPLC chromatogram showing the multi-phospholipid composition of 40% non-purified soy PS.

FIG. 1B is a phase contrast optical microscope micrograph showing aggregates of liposomes, when NSPS (40% PS) is condensed with calcium cation. Note that no cochleates formed.

FIG. 2A is an HPLC chromatogram of a purified PSPS showing a high content of PS (Rt=3.456).

FIG. 2B is an electron micrograph after freeze fracture showing a cross section of a cochleate formed with PSPS. Note the bilayer shape.

FIG. 2C is a micrograph of a cochleate cylinder present in the same preparation.

FIG. 3 is a photomicrograph showing cochleate cylinders as rolled up bilayers.

### DETAILED DESCRIPTION OF THE INVENTION

The following terms when used herein will have the definitions given below.

A “cochleate” is a stable, phospholipid-cation precipitate that can be either empty or loaded.

An “empty cochleate” is a cochleate that is comprised only of phospholipid and cations.

A “loaded cochleate” is a cochleate that has one or more bioactive compounds within the phospholipid-cation structure.

“Soy phosphatidylserine” is phosphatidylserine that has been derived from a soy based composition.

“Polyene” refers to any polyene antibiotic or antifungal agent. Preferred polyenes include nystatin and amphotericin-B.

In practicing the present invention improved phospholipid based cochleates are made by using soy phosphatidylserine in an amount of at least about 75% by weight of the lipid component of the cochleates. Alternatively, the soy phosphatidylserine can be about 80% or 90% by weight or more of the lipid component of the cochleate. In a preferred embodiment the phospholipid is substantially 100% soy phosphatidylserine.

Phosphatidic acid is a preferred phospholipid when there is an additional phospholipid besides phosphatidylserine in the presently improved cochleates. Other phospholipids in addition to phosphatidic acid that can be used in the presently improved cochleates include phosphatidylcholine, phosphatidylinositol and phosphatidylglycerol. Mixtures of the additional phospholipids can also be used in combination with the soy phosphatidylserine.

The soy phosphatidylserine starting material is made by purifying soy phospholipid compositions, which are mixtures of several soy phospholipids, according to well known and standard purification techniques. Purified soy phosphatidylserine is also a commercially available product.

The present cochleates are made by standard cochleate preparation techniques where soy phosphatidylserine is used in an amount of at least about 75% by weight of the lipid component of the cochleate. The cochleates can be empty or loaded with a bioactive agent. Typically, liposomes are formed employing standard well known procedures and then a multivalent compound is mixed with the liposomes whereby the cochleates precipitate and form.

Any multivalent compound can be used to precipitate the cochleates from the liposome starting materials. Preferably, the multivalent compounds are divalent cations such as for example Ca⁺⁺, Zn⁺⁺ and Mg⁺⁺. Preferred sources of these cations include the chloride salts of calcium, zinc and magnesium. CaCl₂ is a particularly preferred source of divalent cations.

In one embodiment the present soy phosphatidylserine cochleates are made by a process which comprises the steps of:

(a) preparing small, unilamellar liposomes in an aqueous medium having a pH of between about 10 and about 12 wherein the liposomes have (i) a lipid bilayer comprising soy phosphatidylserine in an amount of at least about 75% by weight of the lipid bilayer and optionally (ii) a load of one or more bioactive compounds;

(b) adding a multivalent cation to the liposomes of (a) to form the soy phosphatidylserine cochleates;

(c) adjusting the pH of the medium to about neutral; and

(d) collecting the soy phosphatidylserine cochleates.

Loaded cochleates made by this process preferably contain amphotericin-B as the drug (load) and calcium as the multivalent cation. The cochleates can contain substantially 100% by weight soy phosphatidylserine as the lipid component or optionally a mixture of phosphatidylserine and up to about 25% by weight phosphatidic acid.

In another embodiment the improved cochleates of the present invention are nanocochleates and can be prepared employing the procedures disclosed in U.S. Pat. No. 6,153,217 which is incorporated herein by reference. This method for producing soy phosphatidylserine cochleates comprises the steps of:

(a) preparing small, unilamellar liposomes in an aqueous medium having a pH of between about 10 and about 12 wherein the liposomes have (i) a lipid bilayer comprising soy phosphatidylserine in an amount of at least about 75% by weight of the lipid bilayer and optionally (ii) a load of one or more bioactive compounds;

(b) mixing the liposomes with a first water soluble polymer to form a suspension;

(c) adding the liposome/polymer suspension into a suspension comprising a second water soluble polymer wherein the first and second polymers are immiscible thereby creating a two-phase polymer system;

(d) adding a multivalent cation to the two-phase polymer system to form the soy phosphatidylserine cochleate; and

(e) collecting the soy phosphatidylserine/polyene cochleate.

The first polymer (Polymer A) and second polymer (Polymer B) used to make the present soy phosphatidylserine cochleates can be of any biocompatible polymer classes that can produce an aqueous two-phase system. For example, polymer A can be, but is not limited to, dextran 200,000-500,000, polyethylene glycol (PEG) 3,400-8,000; polymer B can be, but is not limited to, polyvinylpyrrolidone (PVP), polyvinylalcohol (PVA), Ficoll 30,000-50,000, polyvinyl methyl ether (PVMB) 60,000-160,000, PEG 3,400-8,000. The concentration of polymer A can range from between 2-20% w/w as the final concentration depending on the nature of the polymer. The same concentration range can be applied for polymer B. Examples of suitable two-phase systems are Dextran/PEG, 5-20% w/w Dextran 200,000-500,000 in 4-10% w/w PEG 3,400-8,000; Dextran/PVP 10-20% w/w Dextran 200,000-500,000 in 10-20% w/w PVP 10,000-20,000; Dextran/PVA 3-15% w/w Dextran 200,000-500,000 in 3-15% w/w PVA 10,000-60,000; Dextran/Ficoll 10-20% w/w Dextran 200,000-500,000 in 10-20% w/w Ficoll 30,000-50,000; PEG/PVME 2-10% w/w PEG 3,500-35,000 in 6-15% w/w PVME 60,000-160,000.

The bioactive agent/drug (referred to as “load” or drug) can be hydrophobic in aqueous media, hydrophilic or amphiphilic. The drug can be, but is not limited to, a protein, a small peptide, a bioactive polynucleotide, an antiviral agent, an anesthetic, an anti-infectious agent, an antifungal agent, an anticancer agent, an immunosuppressant, a steroidal anti-inflammatory, a nutritional supplement, an herbal product, a vitamin, a non-steroidal anti-inflammatory, a tranquilizer or a vasodilatory agent. Examples include Amphotericin B, acyclovir, adriamycin, vitamin A, cabamazepine, melphalan, nifedipine, indomethacin, naproxen, estrogens, testosterones, steroids, phenytoin, ergotamines, cannabinoids rapamycin, propanidid, propofol, alphadione, echinomycine, miconazole nitrate, teniposide, taxanes, paclitaxel, and taxotere.

The drug can be a polypeptide such as cyclosporin, angiotensin I, II and III, enkephalins and their analogs, ACTH, anti-inflammatory peptides I, II, III, bradykinin, calcitonin, b-endorphin, dinorphin, leucokinin, leutinizing hormone releasing hormone (LHRH), insulin, neurokinins, somatostatin, substance P, thyroid releasing hormone (TRH) and vasopressin.

The drug can be an antigen, but is not limited to a protein antigen. The antigen can also be a carbohydrate or DNA. Examples of antigenic proteins include envelope glycoproteins from influenza or Sendai viruses, animal cell membrane proteins, plant cell membrane proteins, bacterial membrane proteins and parasitic membrane proteins.

The antigen is extracted from the source particle, cell, tissue, or organism by known methods. Biological activity of the antigen need not be maintained. However, in some instances (e.g., where a protein has membrane fusion or ligand binding activity or a complex conformation which is recognized by the immune system), it is desirable to maintain the biological activity. In these instances, an extraction buffer containing a detergent which does not destroy the biological activity of the membrane protein is used. Suitable detergents include ionic detergents such as cholate salts, deoxycholate salts and the like or heterogeneous polyoxyethylene detergents such as Tween, BRIG or Triton.

Utilization of this method allows reconstitution of antigens, more specifically proteins, into the liposomes with retention of biological activities, and eventually efficient association with the cochleates. This avoids organic solvents, sonication, or extreme pH, temperature, or pressure all of which may have an adverse effect upon efficient reconstitution of the antigen in a biologically active form.

The presently improved cochleates can include loads with multiple antigenic molecules, biologically relevant molecules or drug formularies as appropriate.

The formation of small-sized cochleates (with or without drugs) is achieved by adding a positively charged molecule to the aqueous two-phase polymer solution containing liposomes. In the above procedure for making cochleates, the positively charged molecule can be a polyvalent cation and more specifically, any divalent cation that can induce the formation of a cochleate. In a preferred embodiment, the divalent cations include Ca⁺⁺, Zn⁺⁺, Ba⁺⁺ and Mg⁺⁺ or other elements capable of forming divalent ions or other structures having multiple positive charges capable of chelating and bridging negatively charged lipids. Addition of positively charged molecules to liposome-containing solutions is also used to precipitate cochleates from the aqueous solution.

To isolate the cochleate structures and to remove the polymer solution, cochleate precipitates are repeatedly washed with a buffer containing a positively charged molecule, and more preferably, a divalent cation. Addition of a positively charged molecule to the wash buffer ensures that the cochleate structures are maintained throughout the wash step, and that they remain as precipitates.

The medium in which the cochleates are suspended can contain salt such as sodium chloride, sodium sulfate, potassium sulfate, ammonium sulfate, magnesium sulfate, sodium carbonate. The medium can contain polymers such as Tween 80 or BRIG or Triton. The drug-cochleate is made by diluting into an appropriate pharmaceutically acceptable carrier (e.g., a divalent cation-containing buffer).

The cochleate particles can be enteric. The cochleate particles can be placed within gelatin capsules and the capsule can be enteric coated.

The skilled artisan can determine the most efficacious and therapeutic means for effecting treatment practicing the instant invention. Reference can also be made to any of numerous authorities and references including, for example, “Goodman & Gillman's, The Pharmaceutical Basis for Therapeutics”, (6.sup.th Ed., Goodman et al., eds., MacMillan Publ. Co., New York, 1980).

The improved soy phosphatidylserine cochleates of the present invention containing a bioactive load are conveniently administered to patients orally whereby the cochleates are absorbed into the bloodstream and the bioactive loads are delivered systemically. This is a particular advantage for water insoluble drugs such as amphotericin-B and paclitaxel. Additionally, the toxicity of many hydrophobic drugs is substantially reduced as seen with soy phosphatidylserine cochleates containing amphotericin-B as the load.

In a preferred embodiment of the present invention, a mixture of soy phospholipids containing 90% by weight phosphatidylserine is dissolved in chloroform and then mixed with amphotericin-B dissolved in methanol. The mixture is dried to a film and then hydrated with de-ionized water to make a concentration of about 10 mg phospholipid/mL. The hydrated suspension is sonicated until no liposomes are visible under a 100× microscope lens. Any amphotericin-B crystals that remain are dissolved by adding a base such as NaOH. Cochleates are formed by the slow addition of CaCl₂ to the suspension of liposomes at a molar ratio of lipid to Ca²⁺ of about 1:1. The pH is then adjusted to neutral with an acid.

In another preferred embodiment, a mixture of soy phospholipids containing 90% by weight phosphatidylserine is dissolved in chloroform and then mixed with amphotericin-B dissolved in methanol. The mixture is dried to a film and then hydrated with de-ionized water to make a concentration of about 10 mg phospholipid/mL. The hydrated suspension is sonicated until no liposomes are visible under a 100× microscope lens. Any amphotericin-B crystals that remain are dissolved by adding a base such as NaOH to raise the pH of the liposome mixture to between 10-12. The liposome suspension is then mixed with a first aqueous polymer, such as, for example, dextran-500,000, and then injected into a second aqueous polymer, such as, for example, PEG-8000, wherein the first and second polymers are immiscible with each other. CaCl₂ is added to the immiscible polymeric suspension with stirring to form the cochleates. The cochleates are washed with a buffer solution and collected.

The following examples illustrate the practice of the present invention but should not be construed as limiting its scope.

### EXAMPLE 1

Attempt to Prepare Empty Cochleates from Non-Purified Soy PS

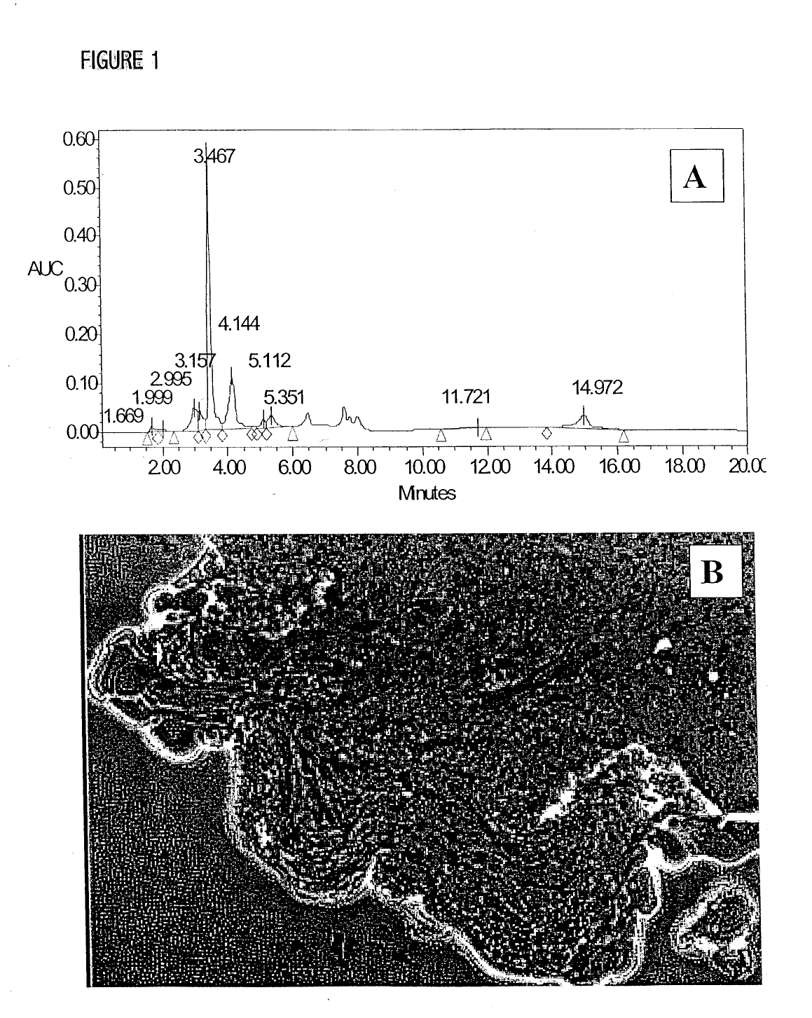

To 50 mg of soy PS (Leci-PS, Lucas Meyer, 40% PS), 5 ml of sterile water was added. The mixture was vortexed thoroughly for 3 min. to form liposomes. To 1 ml of the liposome suspension 0.1 ml of CaCl₂ (0.1 M) at a molar ratio of lipid:Ca⁺⁺ of 1:1 was added dropwise. Phase contrast optical microscopy shows the formation of aggregates of liposomes with some domains that move suggesting liposomes swimming around (FIG. 1B). No cochleates were observed. Composition analysis of the lipid used in this preparation was performed using HPLC equipped with a diol column and a gradient mobile phase (A: CHCl3/MeOH/NH4OH 800/145/5, B: CHCl3/MeOH/H2O 600/340/50). HPLC chromatogram showed that soy PS contains more than 11 different compounds with a low percentage of PS (FIG. 1A).

### EXAMPLE 2

Preparation of Empty Cochleates from Purified Soy PS (90%)

Purified soy derived phosphatidylserine (ALC PS 90P) powder was dispersed in sterile water at a concentration of 10 mg of lipid/ml. The suspension was then vortexed for 1 minute followed by sonication for 1 minute. Cochleates were formed by the slow addition (10 μl) of calcium chloride (0.1 M) to the suspension of liposomes at a molar ratio of lipid to calcium of 1:1 and then stored at 4° C. in the absence of light. The structure of empty cochleates was confirmed by transmission electron microscopy after freeze fracture.

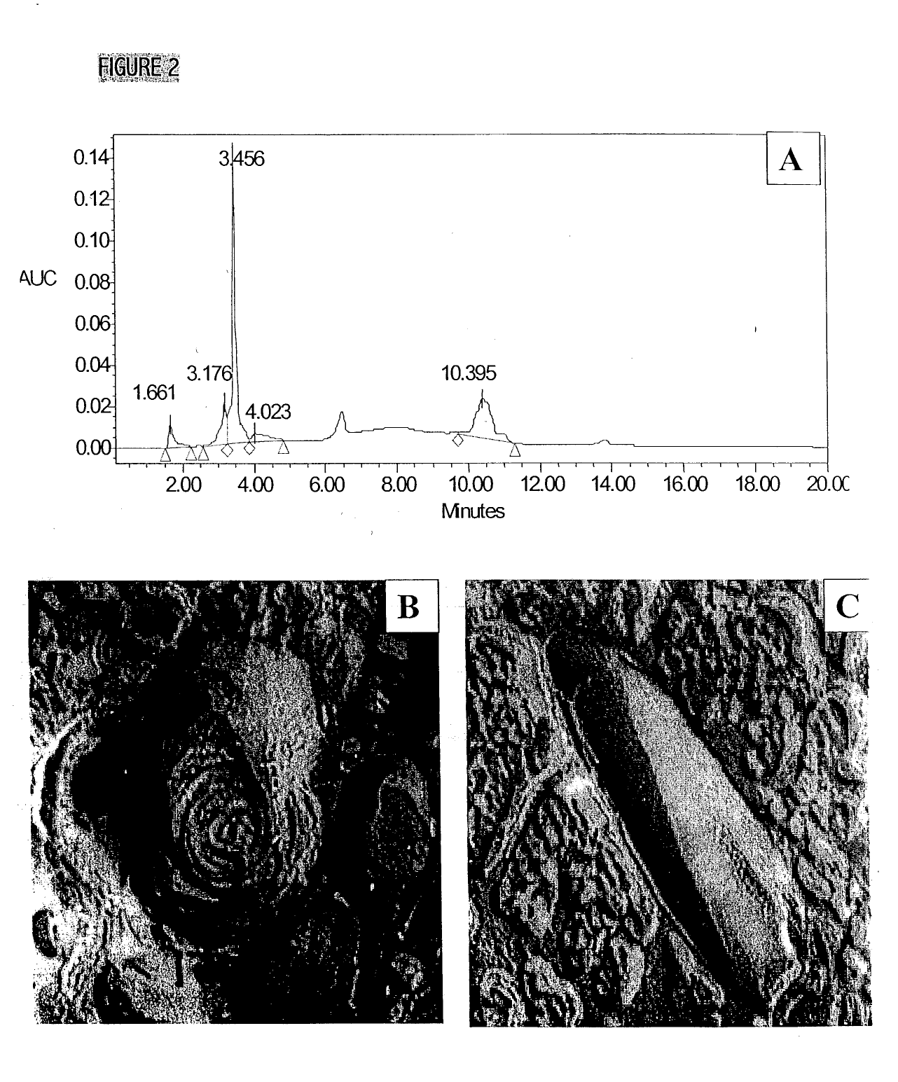

Freeze fracture was performed as follows: Aliquots of each sample were mixed with glycerol to achieve a final concentration of 25% (v/v). A drawn Pasteur pipette was used to apply a small droplet of these suspensions onto a flat-top gold support disc. Rapid sample freezing was achieved by plunging the discs into liquid freon. After 3-4 seconds, the sample was transferred onto a specimen table immersed in liquid nitrogen, prior to insertion into the freeze-fracture apparatus (Balzars, BAF400). Fracturing was carried out at −110° C. and <2×10⁻⁶ mbar, immediately followed by obliquely shadowing with platinum at 45° and application of an electron-translucent carbon backing at 90°. Replicas of samples were removed by submersion in distilled water and subsequently cleaned with commercial bleach solution. Washed replicas were then transferred to grids. Micrographs were obtained using a Zeiss EM 10C Transmission Electron Microscope. FIG. 2B shows the formation of cochleate cylinders characterized by rolled-up bilayers. FIG. 2C is a micrograph of a cochleate cylinder present in the same preparation. Analysis of the lipid by HPLC using the column and gradient used for Example 1 shows that PS has a higher purity than the lipid used in Example 1 (FIG. 2A). PS concentration is around 90%.

### EXAMPLE 3

Preparation of Empty Cochleates from Purified Soy PS (100%)

Purified soy PS (Phosphatidylserine) powder was dispersed in sterile water at a concentration of 10 mg of lipid/ml. The suspension was then vortexed for 1 minute followed by sonication for 1 minute. The cochleates were formed by the slow addition (10 μl) of calcium chloride (0.1 M) to the suspension of liposomes at a molar ratio of lipid to calcium of 1:1 and then stored at 4° C. in the absence of light. The structure of empty cochleates was confirmed by phase contrast optical microscopy and transmission electron microscopy after freeze fracture employing the procedures described in Example 2.

Optical microscopy shows the formation of cochleate aggregates. Cochleates transform into liposomes upon addition of EDTA.

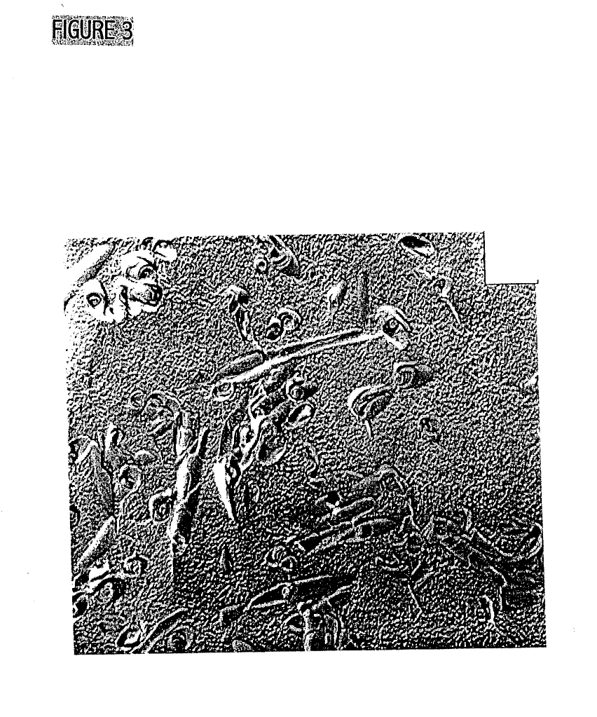

FIG. 3 shows the formation of cochleate cylinders characterized by rolled-up bilayers.

### EXAMPLE 4

Preparation of Amphotericin B-Loaded Cochleates Precipitated with Calcium, Using 90% PS and a High pH Trapping Method

A mixture of soy phosphatidylserine (ALC PS, 90%) in chloroform (10 mg/ml) and AmB (amphotericin-B) in methanol (0.5 mg/ml) at a molar ratio of 10:1 was placed in a round-bottom flask and dried to a film using a Buchi rotavapor at 35° C. The following steps were carried out in a sterile hood. The dried lipid film was hydrated with deionized water at the concentration of 10 mg lipid/ml. The hydrated suspension was purged and sealed with nitrogen, then sonicated in a cooled bath sonicator. Sonication was continued for 10 minutes until there were no liposomes apparently visible under a microscope with a 100× lens. Some AmB crystals remained in the suspension. The pH of the suspension was raised by adding NaOH (1N) until no more AmB crystals were seen. The cochleates were formed by the slow addition of CaCl₂ (0.1 M) to the suspension of liposomes at a molar ratio of lipid to Ca²⁺ of 1:1 and then stored at 4° C. in the absence of light. The pH was adjusted to 7 by addition of HCl 1N. Optical microscopy using phase contrast technique showed the formation of characteristic cochleate aggregates which open to liposomes upon addition of EDTA.

### EXAMPLE 5

Preparation of Amphotericin B-Loaded Hydrogel-Isolated Cochleates Using soy PS (90%)

Step 1: Preparation of Small Unilamellar AmB-Loaded, Vesicles from ALC PS 90P

A mixture of ALC PS (90% soy phosphatidylserine) in chloroform (10 mg/ml) and AmB in methanol (0.5 mg/ml) at a molar ratio of 10:1 was placed in a round-bottom flask and dried to a film using a Buchi rotavapor at 35° C. The following steps were carried out in a sterile hood. The dried lipid film was hydrated with sterile water at the concentration of 10 mg lipid/ml. The hydrated suspension was purged and sealed with nitrogen, then sonicated in a cooled bath sonicator. Sonication was continued for 10 minutes until there were no liposomes apparently visible under a microscope with a 100× lens and a few AmB crystals could be seen. The pH was then raised to 10-11 with 1N NaOH until the crystals disappeared. Laser light scattering (N4 plus) indicated that the AmB liposome mean diameter was 91.7±38.3 nm.

Step 2: Preparation of AmB-Loaded Hydrogel-Isolated Cochleates

The liposome suspension obtained in Step 1 was then mixed with 40% w/w dextran-500,000 in a suspension of 3/1 v/v Dextran/liposome. This mixture was then injected via a syringe into 15% w/w PEG-8,000 [PEG 8000/(suspension A)] under magnetic stirring to result in suspension B. The rate of the stirring was 800-1,000 rpm. A CaCl₂ solution (100 mM) was added to the suspension to reach the final molar ratio of Ca²⁺/DOPS 1:1.

Stirring was continued for one hour, then a washing buffer containing 1 mM CaCl₂ and 150 mM NaCl was added to suspension B at the volumetric ratio of 1:1. The suspension was vortexed and centrifuged at 3000 rpm, 2-4° C., for 30 min. After the supernatant was removed, additional washing buffer was added at the volumetric ratio of 0.5:1, followed by centrifugation under the same conditions. The resulting pellet was reconstituted with the same buffer to the desired concentration. Yellow nanocochleates containing AmB were formed.

What is claimed is:

1. A lipid based cochleate which comprises:
a. a purified soy-based phospholipid that comprises at least about 75% by weight soy phosphatidylserine, and
b. a multivalent cation.

2. The cochleate of claim 1 which is an empty cochleate.

3. The empty cochleate of claim 2 wherein the phospholipid is a mixture of soy phosphatidylserine and phosphatidic acid.

4. A lipid based, loaded cochleate which comprises:
a. a purified soy-based phospholipid that contains at least about 75% by weight soy phosphatidylserine,
b. a multivalent cation, and
c. a bioactive load.

5. The cochleate of claim 4 wherein the bioactive load is at least one member selected from the group consisting of a protein, a small peptide, a polynucleotide, an antiviral agent, an anesthetic, an antibiotic, an antifungal agent, an anticancer agent, an immunosuppressant, a steroidal anti-inflammatory agent, a non-steroidal anti-inflammatory agent, a tranquilizer, a nutritional supplement, an herbal product, a vitamin and a vasodilatory agent.

6. The cochleate of claim 5 wherein the bioactive load is at least one member selected from the group consisting of Amphotericin B, acyclovir, adriamycin, cabamazepine, melphalan, nifedipine, indomethacin, naproxen, estrogens, testosterones, steroids, phenytoin, ergotamines, cannabinoids, rapamycin, propanidid, propofol, alphadione, echinomycine, miconazole nitrate, teniposide, a taxane, paclitaxel, and taxotere.

7. The cochleate of claim 6 wherein the bioactive load is amphotericin B and the multivalent cation is Ca⁺⁺.

8. The cochleate of claim 4 wherein the bioactive load is selected from the group consisting of a polypeptide or an antigen.

9. The cochleate of claim 1 wherein the multivalent cation is zinc or calcium.

10. The cochleate of claim 1 wherein the purified soy-based phospholipid comprises at least about 80% by weight soy phosphatidylserine.

11. The cochleate of claim 10 wherein the purified soy-based phospholipid comprises at least about 90% by weight soy phosphatidylserine.

12. A lipid based cochleate which comprises:
a. at least about 80% by mole soy phosphatidylserine,
b. up to about 20% by mole of a mixture of one or more lipids other than phosphatidylserine, and
c. a multivalent cation.

13. The cochleate of claim 12 wherein one or more of the lipids other than phosphatidylserine is a negatively charged lipid.

14. The cochleate of claim 13 wherein the negatively charged lipid is phosphatidic acid.

15. The cochleate of claim 12 wherein one or more of the lipids other than phosphatidylserine is another phospholipid.

16. The cochleate of claim 12 wherein one or more of the lipids other than phosphatidylserine is selected from the group consisting of phosphatidylcholine, phosphatidylinositol, phosphatidylglycerol and phosphatidylethanolamine.

17. In a cochleate composition containing a lipid and a multivalent cation the improvement which comprises employing soy phosphatidylserine for at least about 75% by weight of the lipid.

18. The improved cochleate of claim 17 wherein at least about 80% by weight of the lipid is soy phosphatidylserine.

19. The improved cochleate of claim 17 wherein at least about 90% by weight of of the lipid is soy phosphatidylserine.

20. The improved cochleate of claim 17 wherein the multivalent cation is zinc, magnesium or calcium.

21. A method for producing soy phosphatidylserine/polyene cochleates which comprises the steps of:
a. preparing small, unilamellar liposomes in an aqueous medium having a pH of between about 10 and about 12 wherein the liposomes have (i) a lipid bilayer comprising soy phosphatidylserine in an amount of at least about 75% by weight of the lipid bilayer and (ii) a load of polyene drug;
b. mixing the liposomes with a first water soluble polymer to form a suspension;
c. adding the liposome/polymer suspension into a suspension comprising a second water soluble polymer wherein the first and second polymers are immiscible thereby creating a two-phase polymer system;
d. adding a multivalent cation to the two-phase polymer system to form the soy phosphatidylserine/polyene cochleate; and
e. collecting the soy phosphatidylserine/polyene cochleate.

22. The method of claim 21 wherein the liposome bilayer contains at least about 80% soy phosphatidylserine.

23. The method of claim 21 wherein step (c), the addition into the second polymer, is done by injection.

24. The method of claim 21 wherein the first polymer is at least one member selected from the group consisting of dextran and polyethylene glycol.

25. The method of claim 24 wherein the first polymer ranges in concentration from 2-20% w/w.

26. The method of claim 21 wherein the second polymer is at least one member selected from the group consisting of polyvinylpyrrolidone, polyvinylalcohol, Ficoll, polyvinyl methyl ether, and polyethylene glycol.

27. The method of claim 26 wherein the second polymer ranges in concentration from 2-20% w/w.

28. The method of claim 21 wherein the two-phase polymer system is at least one member selected from the group consisting of dextran/polyethylene glycol, dextran/polyvinylpyrrolidone, dextran/poly-vinylalcohol, dextran/ficoll, and polyethylene glycol/polyvinyl methyl ether.

29. The method of claim 21 wherein the mulivalent cation is Ca²⁺, Mg⁺⁺ or Zn²⁺.

30. The method of claim 29 wherein the Ca²⁺, Mg⁺⁺ or Zn²⁺ is provided by CaCl₂, MgCl₂ or ZnCl₂.

31. The method of claim 21 wherein the soy phosphatidylserine cochleate is of a particle size of less than about one micron.

32. The method of any of claims 21-31 wherein the polyene drug is amphotericin B.

33. In a method of making a phospholipid based cochleate which comprises employing purified soy phosphatidylserine as the phospholipid wherein the soy phosphatidylserine is at least about 75% by weight of the lipid component of the cochleate.

34. The improved method of claim 33 wherein the soy phosphatidylserine is at least about 80% by weight of the lipid component of the cochleate.

35. A method for producing soy phosphatidylserine/polyene cochleates which comprises the steps of:
a. preparing small, unilamellar liposomes in an aqueous medium having a pH of between about 10 and about 12 wherein the liposomes have (i) a lipid bilayer comprising soy phosphatidylserine in an amount of at least about 75% by weight of the lipid bilayer and (ii) a load of polyene drug;
b. adding a multivalent cation to liposomes of (a) to form the soy phosphatidylserine/polyene cochleates;
c. adjusting the pH of the medium to about neutral; and
d. collecting the soy phosphatidylserine/polyene cochleates.

36. The method of claim 35 wherein the polyene is amphotericin B.

37. A method of treating a patient with a fungal infection which comprises administering to the patient an effective anti-fungal amount of a lipid based cochleate which comprises (i) a phospholipid that contains at least about 75% by weight soy phosphatidylserine, (ii) a multivalent cation and (iii) a polyene anti-fungal agent.

38. The method of claim 37 wherein at least about 90% by mole of the phospholipid is soy phosphatidylserine.

39. The method of claims 37 or 38 wherein the antifungal agent is amphotericin B.
